(19) United States
(12) Patent Application Publication
(10) Pub. No.: US 2009/0071487 A1
(43) Pub. Date: Mar. 19, 2009

(54) SEALING DEVICES

(75) Inventor: John Patrick Keady, Boca Raton, FL (US)

Correspondence Address: PERSONICS HOLDINGS INC., 5100 Town Center Circle, Tower 11, Suite 510, BOCA RATON, FL, 33486, US

(73) Assignee: PERSONICS HOLDINGS INC., Boca Raton, FL (US)

(21) Appl. No.: 12/209,918

(22) Filed: Sep. 12, 2008

Related U.S. Application Data
(60) Provisional application No. 60/971,794, filed on Sep. 12, 2007.

(51) Int. Cl. A61F 11/00; G10K 11/00

(52) U.S. Cl. 128/864; 181/135

(57) ABSTRACT

At least one exemplary embodiment is directed to a deformable sealing section that can be inserted into an orifice causing sealing of the orifice.

### CROSS-REFERENCE TO RELATED APPLICATIONS

This application claims the benefit of U.S. provisional patent application No. 60/971,794 filed on 12 Sep. 2007. The disclosure of which is incorporated herein by reference in its entirety.

### FIELD OF THE INVENTION

The present invention relates to sealing devices and more particularly, though not exclusively, to earpiece devices using expandable sections.

### BACKGROUND OF THE INVENTION

Various earpieces (e.g. headphones, earbuds, behind the ear, hearing aids, and other devices that direct acoustic energy into an acoustic measuring device (e.g., ear)) have been designed for various uses. Many conventional systems have difficulty sealing in the ear canal. Additionally other orifices (e.g., ear canal, veins, arteries, nose, pipes) can benefit from and enhanced sealing system.

### SUMMARY OF THE INVENTION

At least one exemplary embodiment is directed to a sealing element comprising: a first deformable chamber, containing a first medium; and a membrane surrounding the first deformable chamber, where the membrane is configured to expand at a first end of the deformable chamber, while a second end of the deformable chamber is compressed when inserted into an orifice.

At least one exemplary embodiment directed to a sealing element comprising: a first deformable chamber, containing a first medium; a second deformable chamber operatively connected to the first deformable chamber; a first membrane surrounding the first deformable chamber; and a second membrane surrounding the second deformable chamber, where the second deformable chamber is configured to change in volume so that the first deformable chamber is moved.

At least one exemplary embodiment is directed to an earpiece comprising: an instrument package; a flexible acoustic coupler; and a sealing element encasing at least a portion of the acoustic coupler, where the sealing element includes a deformable material that changes viscosity in the response to an applied energy change.

Further areas of applicability of exemplary embodiments of the present invention will become apparent from the detailed description provided hereinafter. It should be understood that the detailed description and specific examples, while indicating exemplary embodiments of the invention, are intended for purposes of illustration only and are not intended to limited the scope of the invention.

### BRIEF DESCRIPTION OF THE DRAWINGS

Exemplary embodiments of present invention will become more fully understood from the detailed description and the accompanying drawings, wherein:

FIG. 1 illustrates a cross section of an orifice (e.g., ear canal, anal, nose, pipe);

FIG. 2 illustrates a sealing device being inserted into an orifice;

FIGS. 3-12 illustrate cross sections of sealing devices in accordance with at least one exemplary embodiment; and

FIGS. 13A, 13B, and 13C illustrate an insertion of a sealing device into an orifice in accordance with at least one exemplary embodiment.

### DETAILED DESCRIPTION OF EXEMPLARY EMBODIMENTS OF THE PRESENT INVENTION

The following description of exemplary embodiment(s) is merely illustrative in nature and is in no way intended to limit the invention, its application, or uses.

Exemplary embodiments are directed to or can be operatively used on various wired or wireless orifice insertion devices (e.g., earbuds, headphones, ear pieces, vein catheters, behind the ear devices or other acoustic devices as known by one of ordinary skill, and equivalents).

Processes, techniques, apparatus, and materials as known by one of ordinary skill in the art may not be discussed in detail but are intended to be part of the enabling description where appropriate. For example specific materials may not be listed for achieving each of the targeted properties discussed, however one of ordinary skill would be able, without undo experimentation, to determine the materials needed given the enabling disclosure herein. Such code is intended to fall within the scope of at least one exemplary embodiment.

Additionally exemplary embodiments are not limited to earpieces, for example some functionality can be implemented on other systems that can be inserted into orifices.

Notice that similar reference numerals and letters refer to similar items in the following figures, and thus once an item is defined in one figure, it may not be discussed or further defined in the following figures.

### Exemplary Embodiments

The materials that can be used within a sealing section is variable. For example a sealing element can have a sealing section that can have chambers, each filled with a different fillable material than a membrane which can be encasing the fillable material. The sealing section can use various materials (e.g., viscosity varying polymers), for example polymers that are liquid at one temperature then gell at another, or switch between a gell and liquid with PH, current, pressure, or any other variation in energy, or any other similar material as known by one of ordinary skill in the relevant arts. For example the following is a non-limiting list of references that discuss materials that can be used: U.S. Pub. No. 2002/0168319; U.S. Pat. No. 6,660,247; U.S. Pat. No. 6,352,682; U.S. Pat. No. 6,113,629; U.S. Pat. No. 6,090,911; U.S. Pat. No. 5,976,648; U.S. Pat. No. 5,942,209; U.S. Pat. No. 5,939,485; U.S. Pat. No. 5,876,741; U.S. Pat. No. 5,858,746; U.S. Pat. No. 5,843,156; U.S. Pat. No. 5,766,704; U.S. Pat. No. 5,749,922; U.S. Pat. No. 5,702,361; U.S. Pat. No. 5,695,480; U.S. Pat. No. 5,674,287; U.S. Pat. No. 5,662,609; U.S. Pat. No. 5,634,946; U.S. Pat. No. 5,589,568; U.S. Pat. No. 5,575,815; U.S. Pat. No. 5,525,334; U.S. Pat. No. 5,514,379; U.S. Pat. No. 5,410,016; U.S. Pat. No. 5,256,765; U.S. Pat. No. 5,252,318; U.S. Pat. No. 5,213,580; U.S. Pat. No. 6,660,247; and U.S. Pat. No. 4,732,930.

Additionally, the fillable material referred to herein can also be viscous and can include silicone-based polymers, gels, vinyl elastomers, or any other material of sufficient properties to allow the deformation of a membrane cavity from user contact. Materials can also be used to provide a slow reformation of the original membrane cavity shape after it has been deformed and released. In this regard, a silicone gel or other non-cross-linked polymer or uncatalyzed materials may be used. It should be appreciated that the composition of the fillable material could be altered for applications in which varied membrane characteristics are desired (i.e. more stiffness, durability, more or less deformability and/or longer-lasting deformation). The fillable material can be elastically deformed or it may be deformed by displacement, which is the actual movement or flow of the fillable material in response to pressure, such as that from a user's fingertips. In addition, the fillable material could be altered for applications in which varied temperature or light conditions would be encountered during the use of particular products on which the membrane cavity is mounted.

If a membrane is used, a portion of a membrane connected to a structure (base membrane) can be made of any material, rigid or elastic, including various plastic or metal materials, or it can be made of a membrane formed of thin rubber-based material, deformable plastic or silicone-based materials or other elastomeric materials suitable for a given application. If the base is configured as a flexible membrane, the cavity can more easily conform to a product's surface, thereby increasing the ease with which the cavity can be installed, removed, and replaced. Likewise, the outer membrane also can be made of a thin rubber-based material, deformable plastic or silicone polymer materials, or other elastomeric materials suitable for a given application. If the base membrane and outer membrane are made of silicone material, both can be from 0.50 mm to 2.5 mm in thickness. In this regard, the base may be a membrane instead of a piece of rigid material. The edges of the outer membrane and the base membrane can be mechanically fastened or clamped forming the membrane cavity. Additionally, at least a portion of the base membrane can be adhesively attached (e.g., adhesive tape, glue) or mechanically fastened to the support structure.

The silicone sealant can be of an acetoxy cure type. In particular, upon exposure to moisture, the silicone sealant will give off small amounts of acetic acid while the sealant cures. It is not recommended that the acetic acid vapors be inhaled. The sealant will cure in 24 hours and has a tack free time of 10-20 minutes at 77.degree. F. (25.degree. C.) with 50% relative humidity. The sealant's tensile strength is approximately 350 psi, its elongation property is 450%, and its hardness is approximately 25-30 Shore A. The sealant has temperature stability from −85.degree. F. to 450.degree. F. (−65.degree. C. to 232.degree. C.) and can withstand intermittent exposure to temperatures as high as 500.degree. F. (280.degree. C.). The sealant is believed to have good resistance to various weathering conditions, including UV radiation, rain, snow, etc, without hardening, cracking, or shrinking.

For optimum adhesion with the above adhesive, the support structure and the lower surface of the base membrane should be clean, dry, and free from oil, grease or other foreign material. If necessary, metal surfaces should be wiped with a non-oily solvent. Rubber surfaces should be abraded to promote adhesion. Depending on environmental conditions, the base and product surface should be joined within 5-10 minutes, before the tack-free time of the sealant passes.

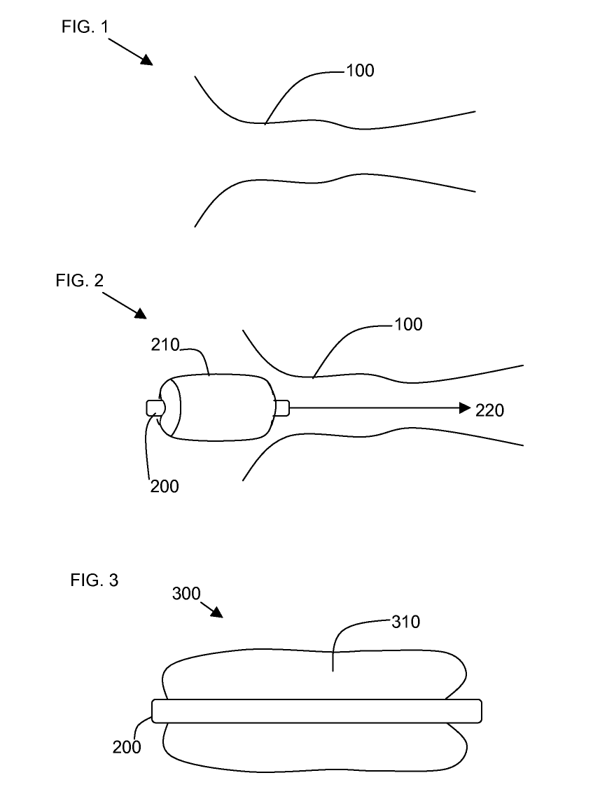

The sealing sections (e.g., 210, 310, 410, 510) can be attached operatively to a support tube (e.g., 200), which can be flexible forming a sealing element (e.g., 300, 400, 500, 600, 700, 800, 900, 1000, 1100, 1200, 1300). The sealing element can be attached to an orifice insertion device that can be inserted (e.g., 220) into an orifice 100 (FIG. 1, FIG. 2).

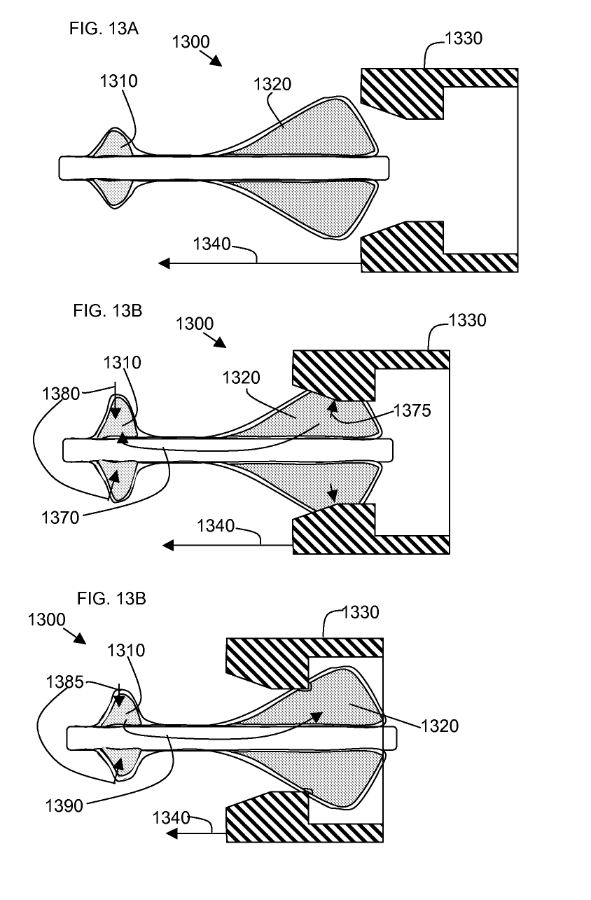

Note that during insertion (e.g., 220) the sealing section (e.g., 210) can be reduced in size (FIGS. 13A, 13B, 13C), have multiple chambers (e.g., 620, 710, 720, 810, 820, 910, 920, 1010, 1020, 1030, 1110, 1120, 1130, 1210, 1220, 1230, 1310, 1320), can be inflated (e.g., FIGS. 11 and 12), can be electro activated (e.g., via PH, current, voltage), or a combination thereof.

FIG. 3 illustrates a cross section of a sealing element including a sealing section 310 and a support tube 200. Note the support tube 200 can be flexible or rigid and made from various materials for example the materials listed for the fillable material, metallic, plastic, rubbers, and polymers. Tube 200 can also be multi-lumen, be sealed at one end, or open at one end, or have tubes within some of which are open and/or closed at one or both ends.

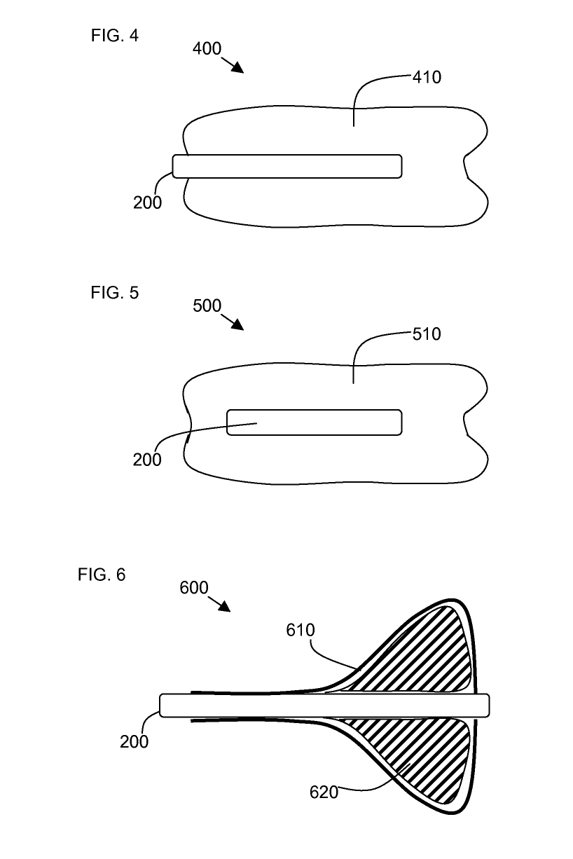

FIG. 4 illustrates a cross section of a sealing element including a sealing section 410 and a support tube 200. Note the support tube 200 can be run through the sealing section (e.g., 300), or partially through (e.g., 400), or completely encompassed by the sealing section (e.g., 500, FIG. 5).

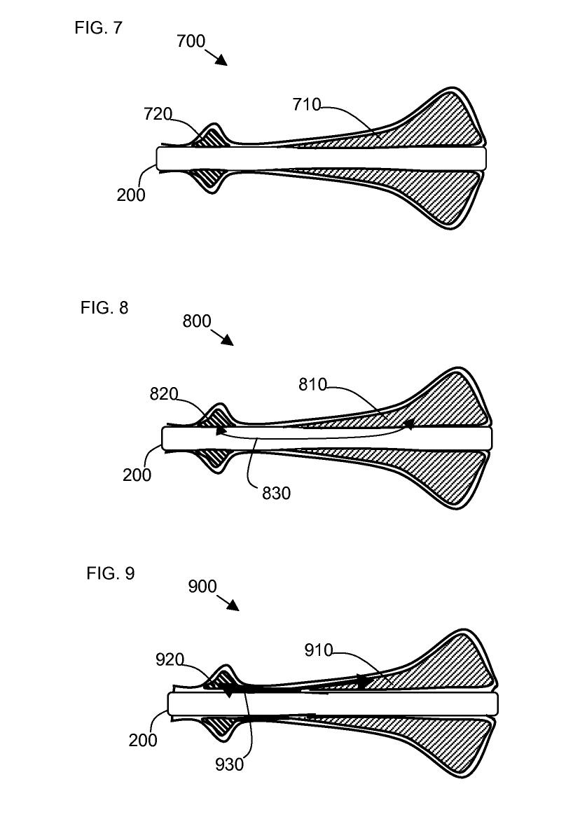

FIG. 6 illustrates a sealing element 600, which includes a membrane 610, and a single chamber 620. Note that various shapes can be used and the shaped illustrated herein are examples only. Exemplary embodiment can include multiple chambers. For example FIG. 7 illustrates a sealing element 700 including two sealing sections (e.g., chambers and membranes 710 and 720). Material can flow through the tube 200 into either or both chambers (see FIG. 11 for an example), or between chambers inside the tube 200 (e.g., 830, FIG. 8), and/or outside the support tube 200 (e.g., 930, FIG. 9). Additionally each chamber can be encompassed with membranes that can deform providing restoring forces opposite to the deformation.

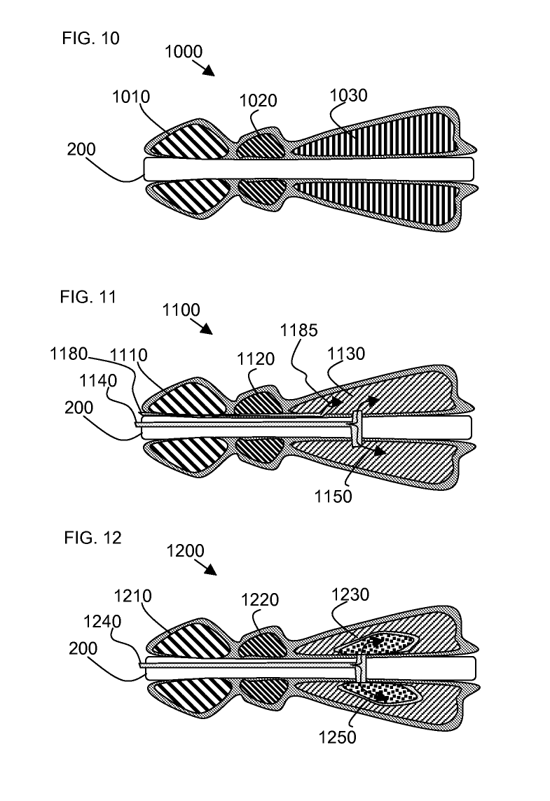

FIGS. 10, 11, and 12 illustrate several sealing elements (e.g., 1000, 1100, 1200) with multiple sealing sections (e.g., 1010, 1020, 1030, 1110, 1120, 1130, 1210, 1220, 1230). Each sealing section can have various fillable material (e.g., gas, water, mineral oil, sweet almond oil, alcohol, air, H, O, and mixtures thereof).

The sealing sections (e.g., 1130) can be filled (e.g., 1150, 1130) or reduced via tubes that can feed from inside 200 (e.g., 1140) or outside 200 (e.g., 1180). Additionally sealing sections (e.g., 1230, 1250) can have multiple chambers some of which can be filled and expanded (or reduced and collapsed) impacting a neighboring section. For example FIG. 12 illustrates a sealing section 1250 being filled which pushes on sealing section 1230. Likewise if sealing section 1250 is reduced then sealing section 1230 will fold closer to tube 200.

FIGS. 13A, 13 B and 13 C illustrate the insertion (e.g., 1340) of a sealing element (e.g., 1300) into an orifice (1330). The sealing section 1320 is deformed upon encountering a smaller cross section during insertion, and if the two sealing sections (1310 and 1320) are coupled, flow 1320 can flow to 1310, expanding 1310. If sealing section 1310 is encompassed by an elastic membrane then a restoring force (e.g., 1380, 1385) opposing the expansion presses back, creating a pressure and/or force (e.g., 1375) against the side of the walls of the orifice (e.g., ear canal). When 1320 is free to expand back to its original state then the expanded 1310 can reduce in size, and the restoring force 1385 can change (e.g., get larger or smaller).

Note that exemplary embodiments can have multi-chambers, multiple membranes that are inflatable and/or deformable. In addition to inflatable configuration variations, the sealing (which could also be stability sections) can be multiple chambers with fillable material inserted, and encased by a membrane. Various types of materials can be used. For example when a force if applied to the membrane the fillable material, which for example can be a gell, can change its viscosity such that the fillable material can flow. Thus, upon an application of a force the gell will partially liquefy and allowed deformation of the section (e.g., during insertion in the ear canal), whereupon there being no force the section re gell in their current deformation, creating a seal.

Note that in at least one exemplary embodiment a sealing element can be formed using several inflatable or deformable bladders which can be operatively connected via several restraining sleeves. The sleeves, which can include an acoustic channel that couples amongst sleeves, can be connected via a fastener, for example a flexible screw. The screw can be bent to allow bending of the sleeves (e.g., the intersection of which can form tube 200).

Note that the sizes of sealing elements can vary depending upon the devices that they are coupled with. For example in an earpiece that is about 10s of mm in diameters, and 10s mm in length, with a mass varying from 5 grams to hundreds of grams, the sealing elements can be of similar size. For example sealing sections can be in the minimal compressed dimension roughly 7 mm (ring diameter), whereas in the uncompressed dimension can be 14 mm (ring diameter). For example at least one exemplary embodiment has a non deformable core diameter of about 5 mm with a length of about 25 mm, with an additional surrounding deformable lay (e.g., sealing section) of an additional 5 mm on either side of the core.

FIG. 8 illustrates an assembly example of another earpiece in accordance with at least one exemplary embodiment. Sleeves (830C, 830B, 830A) retain sealing and/or stability sections (e.g. bladder) 820. In this particular configuration the sleeves screw into each other. If the sleeves are made from a semi flexible material then there can be some bending about the original co-axial position (a line along the center of the acoustic channel, see FIG. 8).

In one configuration an outer membrane contains a fillable material, such as viscosity variable polymers (e.g., that gellify when reaching body temperature) while underneath another membrane encapsulates another medium, which can be a fluid (e.g., liquid, gas) that can be increased or reduced to inflate the inner membrane in the positive/negative radial direction. The medium can be fed via an inflation tube. Note that although two membranes are discussed, more can be used with various levels of inflation and various materials.

Note that a moveable plate can be moved to press a pressure plate, where the moveable plate is operatively connected via connection rods with the pressure plate. As the pressure plate moves in the ear drum direction a deformable bladder can expand. The material in the bladder can be a viscosity varying polymer, which liquidfies when pressure is applied and gellifies when pressure is reduced.

Note that a pressure release mechanism can also be incorporated to control inflation and pressures. For example upon removal of the earpiece, pulling of a knob can open a vent, which equalizes the pressures on either side of the earpiece so that no ear drum deformation occurs by a sealed chamber region upon removal of the earpiece.

Note that various materials can have different frequency damping effects.

Note that although an sealing element (e.g., used in an earpiece) is described herein, other devices that can use various viscosity polymers, inflatable, or sealing elements are also meant to fall within the scope of at least one exemplary embodiment of the present invention, for example a drain plug, a pipe plug, a device for sealing the pipe up to a design pressure at which the gell will liquefy and be released or other sealing or impact type situations.

Additionally although specific numbers may be quoted in the claims, it is intended that a number close to the one stated is also within the intended scope, i.e. any stated number (e.g., 20 mils) should be interpreted to be “about” the value of the stated number (e.g., about 20 mils).

Thus, the description of the invention is merely exemplary in nature and, thus, variations that do not depart from the gist of the invention are intended to be within the scope of the exemplary embodiments of the present invention. Such variations are not to be regarded as a departure from the spirit and scope of the present invention.

What is claimed is:

1. A sealing element comprising:
a first deformable chamber, containing a first medium; and
a membrane surrounding the first deformable chamber, where the membrane is configured to expand at a first end of the deformable chamber, while a second end of the deformable chamber is compressed when inserted into an orifice.

2. The sealing element according to claim 1 further comprising:
a second deformable chamber, containing a second medium, where the first medium can transfer between the first deformable chamber and the second deformable chamber.

3. The sealing element according to claim 2, where the deformation of the second end causes at least a portion of the first medium to flow to the second deformable chamber.

4. The sealing element according to claim 3, where a second portion of the first medium enters the second deformable chamber, expanding the second deformable chamber.

5. The sealing element according to claim 4, where the expanded second deformable chamber provides a restoring force resisting expansion of the second deformable chamber.

6. The sealing element according to claim 5, where an elastic membrane surrounding the second deformable chamber provides the restoring force.

7. A sealing element comprising:
a first deformable chamber, containing a first medium;
a second deformable chamber operatively connected to the first deformable chamber;
a first membrane surrounding the first deformable chamber; and
a second membrane surrounding the second deformable chamber, where the second deformable chamber is configured to change in volume so that the first deformable chamber is moved.

8. The sealing element according to claim 7, where the second deformable chamber is inflated to increase in volume moving the second deformable chamber.

9. The sealing element according to claim 8, where fluid is injected to inflate the second deformable chamber.

10. The sealing element according to claim 9, where the fluid is at least one of air, water, oil, alcohol.

11. An earpiece comprising:
an instrument package;
a flexible acoustic coupler; and
a sealing element encasing at least a portion of the acoustic coupler, where the sealing element includes a deformable material that changes viscosity in the response to an applied energy change.

12. The earpiece according to claim 11, where the instrument package includes at least one of a microphone and a receiver.

13. The earpiece according to claim 11, where the applied energy change is related to an applied force.

14. The earpiece according to claim 13, where the applied force is the force of a user pulling the device out of an ear canal.
